(19) United States
(12) Patent Application Publication
(10) Pub. No.: US 2014/0303419 A1
(43) Pub. Date: Oct. 9, 2014

(54) LINEAR BUTENES FROM ISOBUTANOL

(71) Applicant: LANXESS Deutschland GmbH, Cologne (DE)

(72) Inventors: Matthias Boll, Cologne (DE); Thomas Foellinger, Dormagen (DE)

(21) Appl. No.: 14/352,068

(22) PCT Filed: Oct. 17, 2012
(86) PCT No.: PCT/EP2012/070584

§ 371 Date: Apr. 16, 2014

(30) Foreign Application Priority Data
Oct. 18, 2011 (EP) 11185644.9

(51) Int. Cl. C07C 1/24

(52) U.S. Cl. CPC C07C 1/24
USPC 585/636; 585/640

(57) ABSTRACT

The present invention relates to the preparation of linear butenes from isobutanol which has preferably been obtained from renewable raw materials by microbial and/or fermentation processes.

The present invention relates to the preparation of linear butenes from isobutanol which has preferably been obtained by a microbial and/or fermentation process from renewable raw materials.

Linear butenes are, for the purposes of the present invention, preferably 1-butene and 2-butene (of the latter, both the trans form and also the cis form), particularly preferably 1-butene.

Isobutanol is among the chemical compounds which can be prepared from natural products by targeted biochemical processes and owing to their C4 skeleton attract the interest of the chemical industry. Isobutene, which is an important building block for the synthesis of solvents and for fuel additives such as methyl-t-butyl ether (MTBE) and is also an important raw material in the plastics industry, can be prepared from isobutanol by dehydration. In addition, there is very great interest in linear butenes since this can both be used directly in the polymerization processes of the plastics industry and can also be converted, for example by partial oxidation, into the important raw material butadiene.

GB 576 480 discloses the synthesis of butylene from sec-butyl alcohol at 230° C. in the presence of hydrochloric acid or iron chloride.

The dehydration of isobutanol to form an isobutene/n-butene mixture by means of acids (sulphuric acid/phosphoric acid mixture) in the liquid phase has also been known for a long time, with the proportion of the linear butenes having been very different and being, for example, up to 66% (Canadian Journal of Chemistry (1970), 48(22), 3545-8). However, the total yield of gaseous products of 1545% in these examples was much too low for economical implementation.

To be able to utilize the reaction in an industrially better way, experiments involving applying sulphuric acid to supports such as γ-aluminium oxide and carrying out the reaction using these in a fixed-bed reactor were carried out. Thus, it is stated, for example, in Applied Catalysis, A.: General (2001, 214(2), 251-257) that in the dehydration of isobutanol, the isomer ratio shifts in the direction of the linear butenes when the temperature is increased to above 400° C. There, selectivities of 53.4% of n-butenes to 40% of isobutene (at 400° C.) and 44.7% of n-butenes to 39.8% of isobutene (470° C.) are found. However, the reaction was firstly carried out at very high temperatures which are not very economical. Secondly, the isobutanol was passed through glass wool which, at about 300° C., was very hot before being introduced into the actual reactor. However, based on the experience gained in the context of the present invention, SiO₂ itself has a great influence on the selectivity of the dehydration.

The reaction of isobutanol to form isobutene over solid catalysts without further additions has been described a number of times. Thus, for example, DD 245866 mentions the use of aluminium oxide, SiO₂, CaHPO₄ and AlPO₄ in general terms. However, a possible influence of these catalysts on the isomer ratio is not discussed. A particularly good selectivity in the direction of isobutene is achieved using pure γ-aluminium oxide (US 20100216958A), Industrial embodiments of this have likewise been described (Proceedings—Annual International Pittsburgh Coal Conference (1993), 10th, 1196-9).

All these processes use isobutanol which has not been obtained by preferably microbial or fermentation processes as starting material or usually employ liquid catalysis.

Finally, the influence of a variety of acid catalysts in the reaction of 2-butanol to form butenes was studied in WO 2008/016428 A2 and WO 2008/066581 A1. Here, sulphuric acid, tungstic acid, sulphated zirconium, Amberlyst®15, 13% of Nafion®/SiO₂, CBV3020 E or H-mordenites were used as catalyst. However, 2-butanol is not the preferred starting material since isobutene cannot also be obtained therefrom.

The dehydration of isobutanol obtained by fermentation by means of a BASF AL3996 catalyst, an aluminium oxide having a BET surface area of 200 m²/g, is known from Top. Catal. (2010) 53:1224-1230. However, the main product here is isobutene as raw material for synthetic fuels. Linear butenes are obtained to only a small extent, in particular at low temperatures.

WO 2008/113834 A1 discloses a process for preparing linear n-butenes by dehydration of isobutanol over an Al₂O₃/SiO₂ catalyst.

It was therefore an object of the present invention, proceeding from isobutanol preferably obtained by fermentation or microbially, to develop a catalyst which gives linear butenes, in particular 1-butene, in economical amounts over a long period of time.

The object is achieved by and the present invention provides a process for the dehydration of isobutanol to linear n-butenes, characterized in that TiO₂ having a high BET surface area of >10 m²/g is used as catalyst in a fixed bed. The BET surface area of the TiO₂ catalyst is preferably from 20 to 400 m²/g, particularly preferably from 40 to 200 m²/g, very particularly preferably from 140 to 160 m²/g, The BET surface areas for the purposes of the present invention are determined by adsorption of nitrogen in accordance with DIN 66131.

For the purposes of clarification, it may be remarked that all definitions and parameters mentioned, specified in general terms or specified in preferred ranges are encompassed in any combinations by the scope of the invention.

Isobutanol, preferably from microbial and/or fermentation processes, as are described, for example, in WO 2009/086423A2; WO 2010/151832A1, WO 2010/151525A1, US 2010/0216958A1, US 2007/0092957A1 or DE 10 2008 010 121 A1, is used as starting material. An isobutanol which is obtained from a fermentation or microbial process and can be used for the purposes of the invention is, according to US 2010/0216958A1, Example 1, preferably a mixture containing 98.5-99.9% by weight of isobutanol and 2-0.1% by weight of water, preferably 98.5-99.9% by weight of isobutanol and 1.5-0.1% by weight of water. In a particularly preferred embodiment, not only isobutanol and water but also 3-methyl-1-butanol can be present in an amount of from 0.5 to 1.5% by weight in the mixture to be used according to the invention. This results in mixtures containing 98-99.8% by weight of isobutanol, 0.1-1.9% by weight of 3-methyl-1-butanol and 0.1-1.9% by weight of water, preferably 98.5-99.8% by weight of isobutanol, 0.1-1.75% by weight of 3-methyl-1-butanol and 0.1-1.75% by weight of water.

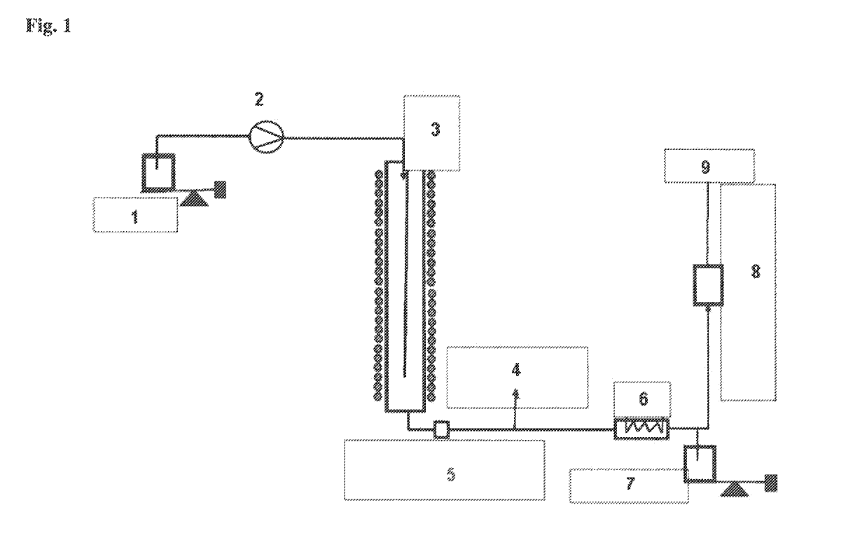

The fixed bed to be used according to the invention is preferably part of a shell-and-tube reactor or multitube reactor which is in turn preferably a constituent of an apparatus shown in FIG. 1. The fixed-bed tube reactor to he used according to the invention is preferably a shell-and-tube reactor or multitube reactor composed of steel or stainless steel. In these reactors, the catalyst is located as a fixed bed in a reaction tube or in a bundle of reaction tubes. The reaction tubes are usually heated indirectly by heating the space surrounding the reaction tubes by combustion of a gas, for example methane. It is advantageous to employ this indirect form of heating only over the first about 20-30% of the length of the fixed bed and heat the remaining length of the bed to the required reaction temperature by means of the radiative heat liberated in the indirect heating. Further types of heating are known to those skilled in the art. Typical internal diameters of the reaction tubes are from 2 to 15 cm. A typical shell-and-tube reactor has about 300-1000 reaction tubes. Such reactors are prior art and are manufactured, for example, by MAN DWE GmbH in Deggendorf, Germany. These have a (stainless) steel perforated plate on which the bed comprising catalyst is placed. According to the invention, the reactor is preferably operated at a pressure of from 1 to 300 bar, particularly preferably from 1 to 100 bar, very particularly preferably from 1 to 10 bar, in particular from 1 to 6 bar and especially very particularly preferably from 3 to 6 bar.

The dehydration of isobutanol in the gas phase in a fixed bed is carried out under conditions which are generally known for this reaction. Preference is given to using temperatures in the range from about 200 to 500° C., preferably from 250 to 400° C., for this purpose. According to the invention, TiO₂ having the above-described BET surface area is used as catalyst in the fixed bed. In particular, TiO₂ having the above-described BET surface area is used. TiO₂ of the anatase type having the abovementioned BET surface areas is especially very particularly preferably used as catalyst. The catalyst geometry is preferably spherical or cylindrical, hollow or solid.

The process of the invention can be operated continuously or batchwise. It is preferably operated continuously.

Industrially, the product mixture can be separated into its individual components as is described, for example, in DE 10 2005 062 700 A1. Here, for example, the by-product isobutene is reacted with an alcohol by means of acid catalysts to form tert-butyl ethers and separated off from the linear butenes before the latter are finally worked up by distillation.

In addition, it has surprisingly been found that the influence of pressure leads to higher selectivity to the product, so that the process is preferably carried out under superatmospheric pressure. In the course of the work leading to the present invention, it was found that increasing the pressure from 1 bara to 5 bara led to an increase in the selectivity to n-butene of up to 4% points. The present invention therefore also provides a method of increasing the selectivities to linear n-butenes, characterized in that the process of the invention is carried out under superatmospheric pressure in the range from 2 to 300 bara, preferably from 2 to 100 bara, particularly preferably from 2 to 50 bara, in particular 5 bara.

The present invention also provides for the use of superatmospheric pressure for increasing the selectivity in the dehydration of isobutanol to linear n-butenes, preferably using a fixed bed of TiO₂ having a BET surface area of >10 m²/g as catalyst.

### Experimental Part

The studies leading to the present invention were carried out in a laboratory apparatus as shown in FIG. 1 and using a laboratory tube reactor having one stainless steel tube having a length of about 1000 mm and a tube cross section of 20 mm. The stainless steel tube had a stainless steel perforated plate. Stainless steel spheres, preferably having a diameter of 2-3 mm, were firstly introduced as space reservers into the steel tube. The catalyst, which consisted of shaped bodies, preferably extrudates or spheres having a length and diameter of about 3 mm, was introduced on top of this bed in order to reduce the pressure drop over the tube. Finally, small rolls of wire meshes, preferably having a diameter and a length of the rolls of about 5 mm, were introduced on top of the catalyst bed up to the upper edge of the reactor tube 3 in FIG. 1. The fixed-bed reactor was installed in an oven which electrically heats the reactor tube along the length from frit (stainless steel perforated plate) to the upper edge of the reactor tube. Installation of five thermocouples in the axis of the tube enabled the internal temperature to be measured above and below the catalyst bed and also at the beginning, in the middle and at the end of the bed. The reactor tube was operated at a pressure of from 1 to 6.5 bar(a), preferably from 3 to 5 bar(a). This could be ensured by installation of an electric pressure regulating valve 5 which was regulated according to the internal pressure and was installed at the hot reactor outlet in FIG. 1. The still hot reaction gas was then taken off and analysed directly in a gas chromatograph 4 in FIG. 1. Sampling was effected via heated capillaries. Separation of the components was effected gas-chromatographically and by means of flame ionization. The apparatus as per FIG. 1 also contained an isobutanol reservoir 1 on a balance, a pump 2 in FIG. 1 for introducing isobutanol into the fixed-bed reactor 3 in FIG. 1, a condenser 6 in FIG. 1 for condensation of water, a balance 7 in FIG. 1 for weighing the water of reaction, a sample cylinder 8 in FIG. 1 for sampling for the off-line gas analysis and the outlet 9 in FIG. 1 from the apparatus to the fume cardboard. For the purposes of the present invention, bar(a) or bara is the “absolute pressure” made up of atmospheric pressure of 1 bar plus the respective gauge pressure. On the subject, see also http://de.wikipedia.org/wiki/Bar (Einheit).

Isobutanol prepared on the basis of crude oil and containing >95% by weight of isobutanol was used as starting material.

### EXAMPLES

The following examples are intended to describe the invention by way of example without restricting it.

### Comparative Example

For comparison, a similar process in which aluminium oxide was used as catalyst is presented.

General set-up:

About 210 mm of stainless spheres (about 2-3 mm diameter) were introduced into a tube made of stainless steel and having a length of about 1000 mm and having a stainless steel perforated plate. The catalyst, which consisted of shaped bodies (for example extrudates having a length and thickness of about 3 mm or spheres having a diameter of about 3 mm), was introduced on top of this bed in order to reduce pressure drop over the tube. Small rolls of fine wire mesh (diameter and length of the rolls is about 5 mm) were then introduced on top of the catalyst bed up to the upper edge of the reactor tube. The reactor was installed in an oven which heated the reactor tube electrically over the length from the frit to the upper edge of the reactor tube. Installation of 5 thermocouples in the axis of the tube enabled the internal temperature to be measured above and below the catalyst bed and also at the beginning, the middle and at the end of the bed. The reactor tube was, for example, operated at atmospheric pressure, under a pressure of 5 tiara or under a pressure of 6.5 bara. This was ensured by installation of an electric valve which was regulated according to the internal pressure and was installed at the hot reactor outlet. The still hot reaction gas was then taken off from the unpressurized gas phase directly downstream of this valve and analysed directly in a gas chromatograph. An Agilent GC 7890 with GICU valve (gas injection valve) from JAS having a heated 250 μl sample loop for analysis was used for these analyses. Sampling was effected by means of heated capillaries made of stainless steel. Separation of the components was effected on a Varian CP-Wax 52CB (25 m*0.25 mm*0.2 μm) with the nonpolar low boilers being diverted by means of a Dean switch to an Agilent HP-AL/KCL (30 m*0.250 mm*5.00 μm) for isomer separation and detection by means of flame ionization. The other components on the CP-Wax were detected in parallel at the flame ionization detector and at the thermal conductivity detector (for the water).

An apparatus as required for the process is shown in FIG. 1. In FIG. 1:

1=Isobutanol reservoir on a balance

2=Pump for introduction of isobutanol into reactor 3

3=Fixed-bed reactor made of stainless steel (1.4571) and having an internal diameter of about 18 mm

4=On-line gas chromatograph

5=Pressure regulating valve

6=Condenser for condensation of water

7=Water of reaction (is weighed)

8=Sample cylinder for sampling for the off-line gas analysis

9=Outlet to fume cupboard

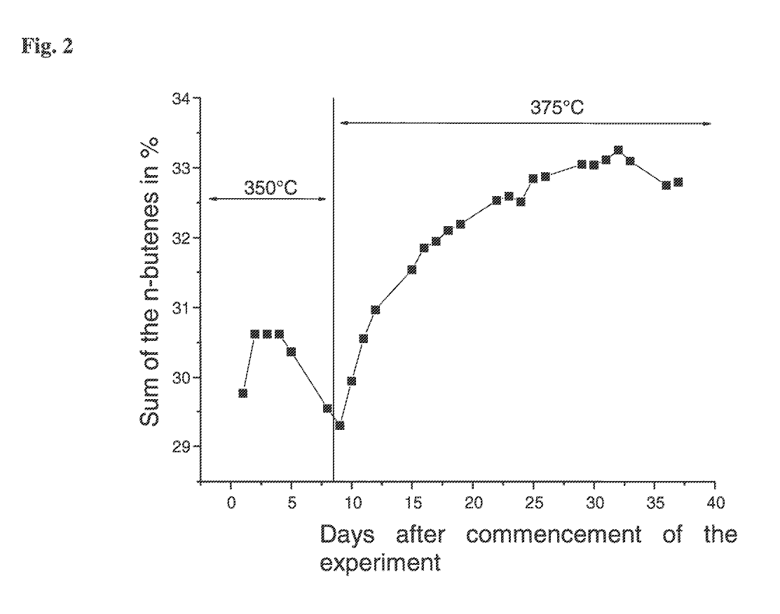

FIG. 2 shows the proportion of the sum of the n-butenes (GC-per cent by area, determined as described in the experimental part) in the reaction product without taking into account the proportions of water in the n-butene formed by the process of the invention from isobutanol in a long-term test using a TiO₂ catalyst from St.-Gobain NorPro (type XT25376). The experimental procedure corresponded to Example 1 in terms of the set-up; the pressure in the reactor tube was left unchanged at 6.5 bar absolute and only the temperature was varied once. FIG. 2 thus shows the results from Example 3 in graph form.

The following catalysts were used according to the invention.

XT25376 from St-Gobain-NorPro, TiO₂ of the anatase type having a BET surface area of about 140 m²/g

ST61120 from St-Gobain NorPro, TiO₂ having a BET surface area of 156 m²/g (CAS 13463-67-7)

It can be seen that the content of n-butenes increases with increasing pressure, so that isobutene and/or n-butene can be prepared as required by the process of the invention. The components can then be separated from one another industrially, as described, for example, in DE 10 2005 062 700 A1.

### Example 1

In a set-up as described in FIG. 1, the reactor tube was charged layerwise in the following order with:
1. 50 ml (21 cm) of stainless steel spheres 1.4571, 3 mm in diameter
2. 93.7 g of catalyst XT25367 from St. Gobain-NorPro (gave a bed height of 52 cm)
3. Steel mesh rings having a diameter of about 5 mm and a height of likewise about 5 mm up to the upper edge of the reactor.

Thermocouples which measure the temperature in the catalyst zone were installed in the bed. The reactor was heated electrically under nitrogen in such a way that the maximum temperature was 350° C. Liquid isobutanol was then introduced from above, and this vaporized in the first zone and arrived in gaseous form and preheated to about 350° C. at the catalyst bed. The amounts introduced are indicated in the following tables. The pressure built up as a result of the reaction in the reactor could be regulated by means of a pressure regulating valve. The reactions at atmospheric pressure and at 5 bara were examined. For this purpose, samples were taken via heated lines from the depressurized product gas atmosphere and these were analysed to determine the gaseous components. The ratio of isobutene to n-butenes was determined by a % by area method, and the total conversion was determined via the unreacted organic constituents in the gas phase and the liquid phase. The total conversion into isobutene and n-butenes was significantly above 95%.

TABLE 1

| Temp | Throughput | Pressure | Isobutene | n-Butene |
|---|---|---|---|---|
| [° C.] | [g/h] | [bara] | [area %] | [area %] |
| 350 | 8.8 | 5 | 63.45 | 29.91 |
| 350 | 9.5 | 1 | 68.03 | 25.92 |
| 350 | 9.5 | 1 | 67.66 | 25.76 |
| 350 | 9.5 | 5 | 63.49 | 28.78 |
| 350 | 9.5 | 5 | 64.00 | 29.35 |

Table 1 shows the dependence of the ratio of n-butene to isobutene as a function of the pressure when using a TiO₂ catalyst XT 25376 from St. Gobain-NorPro using 93.7 g of catalyst (=bed height of the catalyst bed of 52 cm) in a set-up as described in FIG. 1. Furthermore, it can be seen in Table 1 that the pressure dependence of n-butene formation under these conditions is reversible.

### Example 2

In a set-up as described in Example 1, a TiO₂ of the rutile type (XT90045 from St-Gobain NorPro) having a BET surface area of <3 m²/g was installed instead of the XT25376 of the anatase type. A total of 147.7 g of catalyst were used for a catalyst bed of 52 cm.

At a throughput of 7.7 g/h under a pressure of 5 para at 350° C., a total of 151.1 g of isobutanol were introduced and gave a low boiler mixture having a proportion of 77.7% of isobutene and 21.5% as the sum as the n-butenes at a conversion of about 91%.

### Example 3

### Long-Term Test Using XT25376

A catalyst from St-Gobain-NorPro, type: XT 25376 composed of TiO₂ of the anatase type having a BET surface of about 140 m²/g was used as in Example 1. Charging of the reactor as in Example 1. The reactor was operated continually at a pressure of 6.5 para.

Isobutanol was pumped continuously at a rate of about 9 g/h into the reactor tube heated to about 350° C. or 375° C., respectively. After a pressure buildup to 6.5 bara, product was taken off continuously. The results of this experiment are shown in Table 2. Each of the experimental points displays a conversion of >98% based on the isobutanol used.

TABLE 2

| Days after beginning of | Temp. | Throughput | Isobutene | n-Butenes |
|---|---|---|---|---|
| the Experiment | [° C.] | [g/h] | [area %] | [area %] |
| 1 | 350 | 9.5 | 64.46 | 29.77 |
| 2 | 350 | 8.5 | 64.1 | 30.62 |
| 3 | 350 | 8.8 | 64.06 | 30.62 |
| 4 | 350 | 8.8 | 64.06 | 30.62 |
| 5 | 350 | 19.8 | 64.82 | 30.37 |
| 8 | 350 | 3.73 | 61.15 | 29.55 |
| 9 | 375 | 9.1 | 59 | 29.3 |
| 10 | 375 | 9.1 | 58.62 | 29.95 |
| 11 | 375 | 9 | 59.15 | 30.56 |
| 12 | 375 | 9.1 | 58.78 | 30.97 |
| 15 | 375 | 9.1 | 58.09 | 31.54 |
| 16 | 375 | 9 | 58.27 | 31.86 |
| 17 | 375 | 9 | 58.03 | 31.95 |
| 18 | 375 | 9.1 | 58.04 | 32.11 |
| 19 | 375 | 9.1 | 57.93 | 32.2 |
| 22 | 375 | 9.1 | 57.88 | 32.54 |
| 23 | 375 | 9.1 | 57.84 | 32.6 |
| 24 | 375 | 9.1 | 57.69 | 32.52 |
| 25 | 375 | 9 | 57.07 | 32.85 |
| 26 | 375 | 9.1 | 57.49 | 32.88 |
| 29 | 375 | 9.1 | 57.25 | 33.06 |
| 30 | 375 | 9 | 57.33 | 33.05 |
| 31 | 375 | 9.1 | 57.42 | 33.13 |
| 32 | 375 | 9 | 56.58 | 33.27 |
| 33 | 375 | 8.9 | 57.39 | 33.11 |
| 36 | 375 | 9.1 | 57.91 | 32.76 |
| 37 | 175 | 8.8 | 57.81 | 32.81 |

In a set-up as described in Example 1, a TiO₂ type ST61120 from St-Gobain NorPro having a BET surface area of 156 m²/g was installed instead of the XT25376. A total of 83.7 g of catalyst were used for a catalyst bed of 51 cm.

The results for this catalyst are shown in Table 3. The data in respect of conversion are in this case based on the amounts of water of reaction obtained.

TABLE 3

| Temp. | Throughput | Throughput | Conversion | Isobutene | n-Butenes | Water |
|---|---|---|---|---|---|---|
| [° C.] | [g/h] | [g] | [%] | [area %] | [area %] | [g] |
| 350 | 8.6 | 41.6 | ~100 | 55.4 | 26.5 | 11.1 |
| 320 | 8.6 | 165.3 | 84.54 | 63.9 | 29.9 | 34 |
| 290 | 8.6 | 148 | 43.51 | 69.7 | 25.7 | 16.8 |

What is claimed is:

1. Process for the dehydration of isobutanol to linear n-butenes, characterized in that TiO₂ having a bet surface area of >10m²/g is used as catalyst in a fixed bed:

2. Process according to claim 1, characterized in that TiO₂ having a BET surface area of >10m²/g, preferably from 20 to 400 m²/g, is used as catalyst.

3. Process according to claim 1 or 2, characterized in that it is carried out under superatmospheric pressure in order to increase the selectivity to the linear n-butenes.

4. Process according to claim 3, characterized in that superatmospheric pressure means from 2 to 300 bara.

5. Process according to any of claims 1 to 4, characterized in that TiO₂ of the anatase type is used as catalyst.

6. Process according to any of claims 1 to 5, characterized in that the fixed bed is part of a shell-and-tube reactor or multitube reactor.

7. Process according to claim 6, characterized in that the reactor is made of steel or stainless steel.

8. Process according to any of claims 1 to 7, characterized in that it is carried out at temperatures in the range from about 200 to 500° C., preferably from 250 to 400° C.

9. Process according to any of claims 1 to 8, characterized in that the reactor is operated at a pressure of from 1 to 300 bar.

10. Process according to any of claims 1 to 9, characterized in that isobutanol from microbial and/or fermentation processes is used as starting material.

11. Process according to any of claims 1 to 10, characterized in that 1-butene is obtained as n-butene.

12. Process according to any of claims 1 to 11, characterized in that it is operated continuously.

13. Use of a higher pressure for increasing the selectivity in the dehydration of isobutanol to linear n-butenes.

14. Use according to claim 13, characterized in that the dehydration is carried out using TiO₂ having a BET surface area of >10 m2/g as catalyst in a fixed bed.

15. Method of increasing the selectivities to linear n-butenes, characterized in that the dehydration of isobutanol is carried out under a superatmospheric pressure of from 2 to 300 bara, preferably using a TiO₂ catalyst having a BET surface area of >10 m2/g in a fixed bed.
